(19) United States
(12) Patent Application Publication
(10) Pub. No.: US 2013/0322596 A1
(43) Pub. Date: Dec. 5, 2013

(54) IMAGE CAPTURING APPARATUS, IMAGE CAPTURING SYSTEM, METHOD OF CONTROLLING IMAGE CAPTURING APPARATUS, AND STORAGE MEDIUM

(71) Applicant: CANON KABUSHIKI KAISHA, Tokyo (JP)

(72) Inventor: Hitoshi Inoue, Yokohama-shi (JP)

(73) Assignee: Canon Kabushiki Kaisha, Tokyo (JP)

(21) Appl. No.: 13/874,713

(22) Filed: May 1, 2013

(30) Foreign Application Priority Data
Jun. 5, 2012 (JP) 2012-128404

(51) Int. Cl. G01T 1/17; A61B 6/00

(52) U.S. Cl. CPC G01T 1/17; A61B 6/4208
USPC 378/62; 250/394

(57) ABSTRACT

An image capturing apparatus has an image capturing unit that accumulates a charge pixel-by-pixel and outputs an image signal corresponding to the amount of accumulated charge, a generation unit that acquires image signals by performing a set number of readouts of image signals of pixels from the image capturing unit, calculates, with respect to each pixel, image data that is an average of image signals read out from the same pixels, and generates an image having the image data as a characteristic of each pixel, and a correction unit that specifies a position of a defective pixel of the image capturing apparatus and generates correction data that is an image signal that is an average of a set certain number of pixels located in a periphery of a defective pixel.

### BACKGROUND OF THE INVENTION

1. Field of the Invention

The present invention relates to an image capturing apparatus, an image capturing system, a method for controlling an image capturing apparatus, and a storage medium.

2. Description of the Related Art

In order to examine an internal structure by creating an image of the spatial distribution of the intensity of X-rays that pass through a human body or an object, digital images of X-ray images have become widespread. In the medical field in particular, large-sized flat semiconductor image capturing apparatuses (flat panel detectors; below, “FPD”) are used to examine the inner part of a comparatively large object, such as a human body. By using an FPD, it is possible to obtain a digital medical X-ray image with a digitized X-ray intensity distribution can be acquired.

A large amount of superimposed noise on an image is given as a general characteristic of medical X-ray images. This derives from the fact that in order to minimize the X-ray dosage with the goal of reducing radiation exposure to a subject, the X-ray dosage is at a level at which the portion with a weak X-ray intensity to be formed into an image is handled as an energy quantum observed in units of several to hundreds per 1 mm². In order to obtain the maximum amount of information from a medical X-ray image that has a lot of this kind of original noise and use it for a medical examination, it is considered to be necessary to reduce so-called system noise that occurs with an FPD and a peripheral electric circuit as much as possible.

Additionally, a wide dynamic range of the needed X-ray intensity distribution information is given as another characteristic of medical X-ray images. Ordinarily, there are cases where areas in which the X-ray transmission rate of a subject such as a human is low are several thousandths that of areas in which the X-ray transmission rate is high, and if the purpose is to simply examine an image, this can be achieved by saturating the areas with strong X-rays. However, because linearity of the X-ray intensity distribution is needed in order to use it for an arithmetic operation such as X-ray CT, a wide dynamic range is needed.

In order to achieve the two goals of system noise reduction and dynamic range expansion, it is useful to use a sensor that can read out pixel information regarding accumulated charge multiple times as the same voltage value. Specifically, by compositing multiple nondestructive read images, a composite image is created with a smaller amount of noise or an expanded dynamic range. This nondestructive read technique is disclosed in Japanese Patent Laid-Open No. 62-85585 and Japanese Patent No. 2966977.

On the other hand, there is another conventional technique that corrects a defective pixel on an FPD. Because FPDs are manufactured with a semiconductor manufacturing process, defects occur in diodes, transistors, or wiring portions due to accidental events during the manufacturing process or due to the partial intrusion of impurities, and sometimes a specific pixel will not respond to light. This is called a defective pixel, which cannot hold a pixel value obtained from a particular corresponding pixel. Usually, a pixel value that corresponds to a defective pixel is set by estimating it from surrounding normal pixel values. The most typical and mathematically stable estimated value is the average of the peripheral pixels. For example, as shown in FIG. 9, if a pixel p11 is a defective pixel, and peripheral pixels p00, p01, p02, p10, p12, p20, p21, p22 are normal, a corrected defective pixel value p11′ can be calculated with a formula a.

p11′=(p00+p01+p02+p10+p12+p20+p21+p22)÷8 (a)

In another example, as shown in FIG. 10 for example, if a series of pixels p01, p11, and p21 are defective pixels, a corrected defective pixel value p11′ can be calculated with a formula b.

p11′=(p00+p02+p10+p12+p20+p22)÷6 (b)

Defective pixel correction is a technique for outputting a maximum-likelihood value as an average value that corresponds to an unclear defective pixel value, but there is a problem area due to it being an average value. As stated before, because medical x-ray images are generally expressed according to few energy quantum numbers, they are images with a large amount of noise. With defective pixel correction using an average, noise of a pixel value is substantially lessened, but it can become a value that is not valid as a statistical property of an X-ray image. If there is a defective pixel that is isolated singularly, it often does not become a problem, but if defective pixels are successive and become a cluster of defective pixels as shown in FIG. 10, the difference in statistical properties by comparison with surrounding uncorrected pixels is a problem.

As a specific effect of this, for example, if correction is performed when defective pixels form a line, the amount of noise of the corrected line will differ from the amount of noise of the peripheral pixels, and therefore a smooth linear area can be observed by an observer. For example, a case is presumed in which a linear defective pixel cluster, like the one in FIG. 10, exists, and defect correction is carried out by way of the formula b. When the power of random noise (variance value), which is included in a peripheral image, is expressed as σ², the power of the random noise of the defective pixel group is reduced to σ²/6. Because of this, if a linear defective pixel cluster is corrected simply with peripheral average values, the corrected pixel value is a maximum-likelihood value with high reliability, but random noise decreases compared to peripheral pixels. A comparatively smooth line portion becomes prominent and the possibility that it is clearly perceived as a defective pixel by an observer increases.

Regarding this problem, for example, Japanese Patent No. 4532730 discloses operations such as adding fluctuation upon changing the number of peripheral pixels to be averaged with the goal of preserving the statistical properties. However, the method according to Japanese Patent No. 4532730 deviates from a calculation that obtains a maximum-likelihood value with high reliability, which is the primary goal of defective pixel correction, and it is not an accurate defective pixel correction (estimation).

### SUMMARY OF THE INVENTION

In view of the problems stated above, the present invention provides an image capturing technique in which it is possible to correct a defective pixel so that the statistical properties of peripheral pixels and the statistical properties of the defective pixel are the same.

According to one aspect of the present invention, there is provided an image capturing apparatus comprising: an image capturing unit configured to accumulate a charge pixel-by-pixel and output an image signal corresponding to an amount of accumulated charge; a generation unit configured to acquire image signals of pixels from the image capturing unit by a set number of readouts, calculate, with respect to each pixel, image data that is an average of image signals read out from the same pixel, and generate an image having the image data as a characteristic of the pixels; and a correction unit configured to specify a position of a defective pixel of the image capturing unit with use of information indicating a position of a defective pixel, and correct the image data by generating correction data that is an average of image signals of a set certain number of pixels located in a periphery of the defective pixel.

According to the present invention, a defective pixel can be corrected so that the statistical properties of peripheral pixels and the statistical properties of the defective pixel are the same.

Further features of the present invention will become apparent from the following description of exemplary embodiments with reference to the attached drawings.

### BRIEF DESCRIPTION OF THE DRAWINGS

FIG. 1 is a diagram showing an example of a configuration of an image capturing apparatus of an embodiment.

FIG. 2 is a diagram diagrammatically illustrating a calculation performed by an image capturing apparatus of an embodiment.

FIG. 3 is a diagram showing an example configuration of an image capturing apparatus of an embodiment.

FIG. 4 is a diagram illustrating an S/N ratio of an X-ray image captured with an image capturing apparatus of an embodiment.

FIG. 5 is a diagram showing an example of a configuration of an image capturing apparatus of an embodiment.

FIG. 6 is a diagram showing an equivalent circuit of an FPD that can perform nondestructive reading.

FIG. 7 is a diagram showing a configuration of a noise reducing circuit that performs nondestructive reading.

FIG. 8 is a diagram showing a configuration of a dynamic expansion circuit that performs nondestructive reading.

FIG. 9 is a diagram for describing a conventional method of pixel defect correction.

FIG. 10 is a diagram for describing a conventional method of pixel defect correction.

### DESCRIPTION OF THE EMBODIMENTS

Exemplary embodiments of the present invention will be described in detail below with reference to the drawings. However, the constituent elements described in the embodiments are just examples, and the technical scope of the present invention is defined by the scope of the claims and not by the embodiments below.

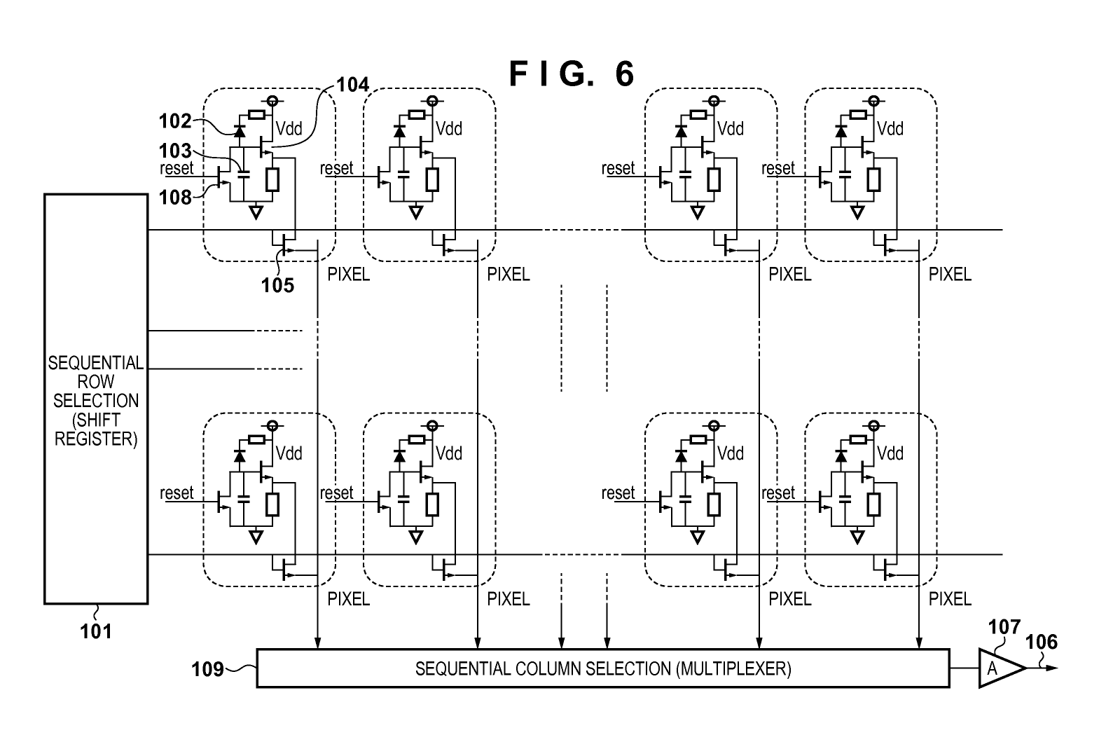

FIG. 6 is a diagram showing an equivalent circuit of a flat panel detector (FPD) that can draw the same voltage multiple times (nondestructive read). The FPD accumulates a charge pixel-by-pixel and outputs an image signal corresponding to the amount of charge accumulated. The areas enclosed by dashed lines are blocks corresponding to one pixel, and the blocks are arranged vertically and horizontally in a matrix pattern. A photodiode 102 and a capacitor 103, which accumulates electric energy obtained by the photodiode 102 as a charge, are connected to a block corresponding to one pixel. The accumulated charge is detected as a voltage in the capacitor 103 and the voltage is output by a source follower amplifier (indicated by reference numeral 104) constituted by a transistor and a load.

A characteristic of this source follower amplifier is that, because the input impedance is high, a charge stored in the capacitor 103 is not consumed. Due to the output of a shift register 101 for sequential row selection being input to the gate of a transistor 105, voltage from the capacitors 103 is transmitted to a multiplexer 109 for sequential column selection.

Voltage data of row 1, selected by the shift register 101, and column 1, selected by the multiplexer 109, is sequentially output as a signal (indicated by reference numeral 106) via an amplifier 107 that holds a gain A. Here, because the source follower amplifier 104 does not consume the charge, it is possible to draw the same voltage multiple times. Because of this, even if the image signals (voltages) of pixels that configure the FPD are read out multiple times, it is possible to read out the same image signal (voltage) each readout time. This kind of readout of an image signal (voltage) is called a “nondestructive read”. If the charge accumulated by the capacitor 103 is not needed, a transistor 108 can reset a block corresponding to one pixel, and return the block to an initial state in which another charge can be accumulated.

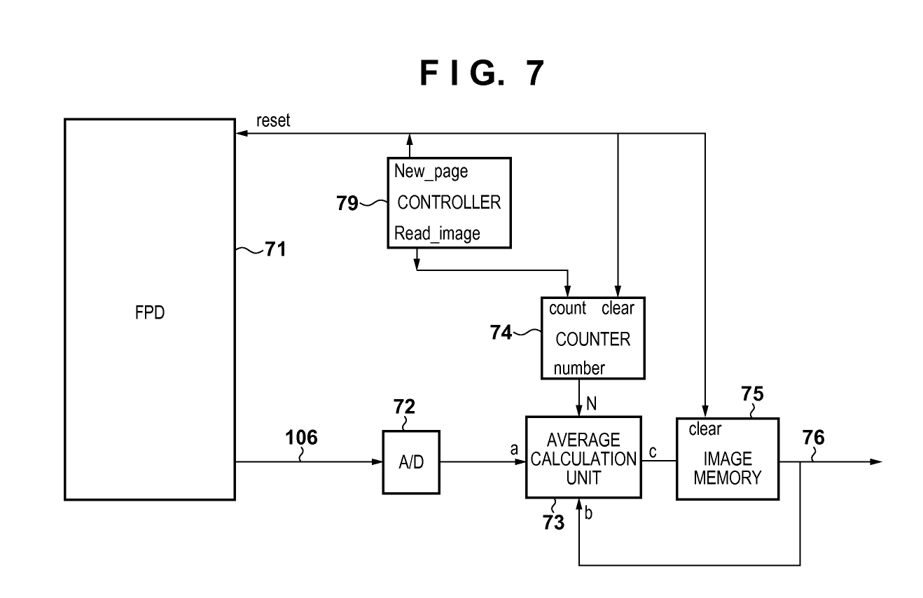

FIG. 7 is a block diagram of an image capturing apparatus that composites data (a nondestructive image), which is read out multiple times using an FPD that can perform a nondestructive read, from blocks corresponding to one pixel each, and generates a composite image with little system noise. An FPD 71 represents the whole FPD shown in FIG. 6. Output voltages from blocks corresponding to one pixel each in the FPD 71 are output as signals indicated by reference numeral 106.

A controller 79 that performs overall control of the image capturing apparatus outputs a New_page signal at a time immediately before a pixel charge is accumulated and resets charges accumulated by blocks of the FPD 71. Additionally, the controller 79 outputs a Read_image signal at a time when a nondestructive read is being performed. Upon receiving the Read_image signal output from the controller 79, the number of times a nondestructive read is performed is counted. A counter 74 counts the number of times a nondestructive read is performed. When the counter 74 receives the New_page signal output from the controller 79, it clears the count value to zero.

An A/D (analog/digital) converter 72 converts the signal 106, which is an output voltage (nondestructive image information) from the FPD 71, into a digital value, and outputs the digital value to an average calculation unit 73. The average calculation unit 73 has two data inputs a and b, and outputs a value (average value) shown as c based on a count number N for obtaining an average. An output value c of the average calculation unit 73 is calculated with an equation 1 written below. The average calculation unit 73 can use a floating-point operation in the calculation of equation 1.

c=b+(a−b)/N(N≧1) (1)

The output value c of the average calculation unit 73 is input to an image memory 75 and stored. When the image memory 75 receives the New_page signal output from the controller 79, all of the contents of the image memory 75 can be cleared. The pixel values stored in the image memory 75 are read out sequentially during the next nondestructive read, and are input to the average calculation unit 73 (data input b). An average value (c) of the nondestructive read image is stored in the image memory 75. An averaged image of the nondestructive read is output as a composite image from the image memory 75 to a signal line 76, and this composite image data with little system noise is obtained.

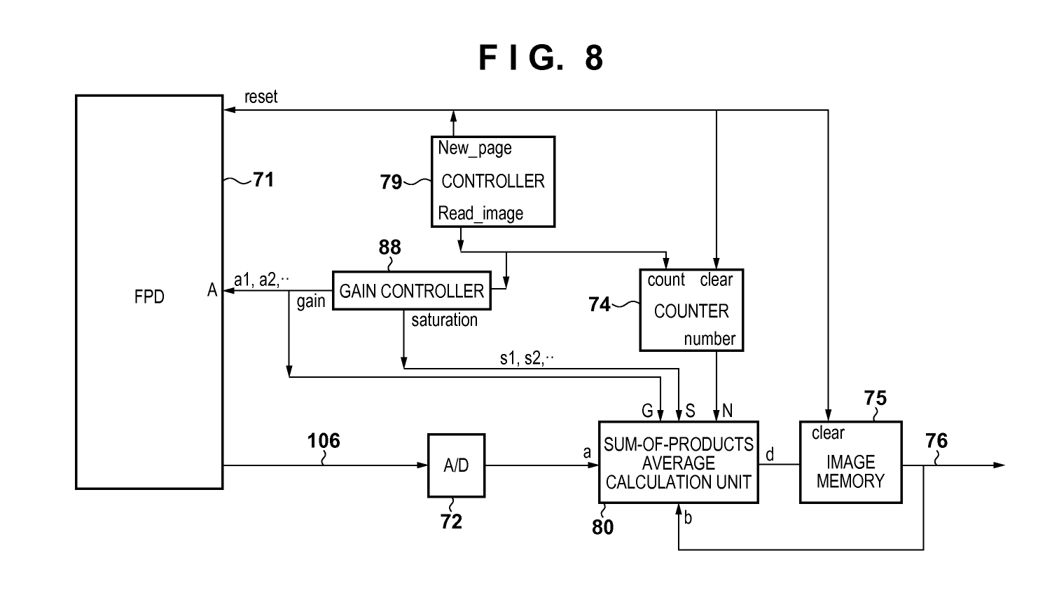

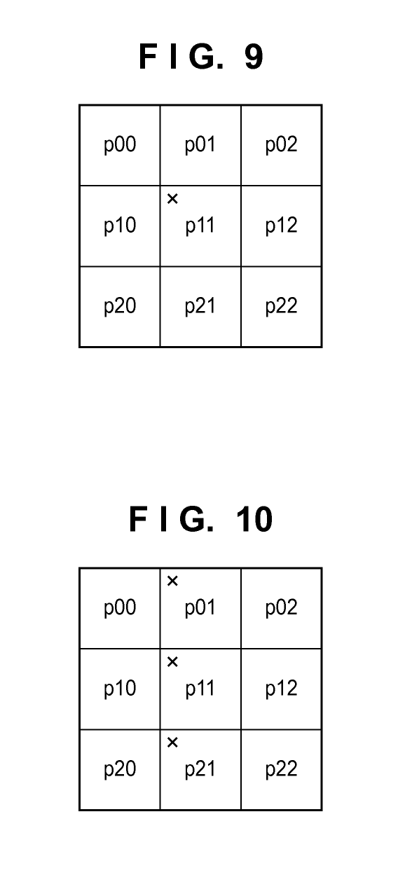

FIG. 8 is a block diagram of the image capturing apparatus that composites data (a nondestructive image), which is read out multiple times using an FPD that can perform a nondestructive read, from blocks corresponding to one pixel each, and generates a composite image with an expanded dynamic range. In FIG. 8, blocks with identical functions to those in FIG. 7 are denoted by identical reference numerals, and descriptions of overlapping functions of blocks will be omitted.

When a gain controller 88 receives the Read_image signal output from the controller 79 at the time of the nondestructive read, it outputs gain values to the FPD 71 at the same time. The gain values have been set in advance at different values such as a1, a2, and the like, and the gain controller 88 outputs these gain values to the FPD 71 via a gain signal line.

In accordance with the gain value output from the gain controller 88, the FPD 71 sets the gain A of the amplifier 107 provided in the last step of output described in FIG. 6. The A/D converter 72 converts the signal 106 and outputs the resulting digital value to a sum-of-product average calculation unit 80. The gain controller 88 holds in advance pixel values s1, s2, . . . , at which the amplifier is saturated and the A/D conversion output cannot be used in conformity with the gain values a1, a2, . . . , and outputs them to the sum-of-product average calculation unit 80. Here, “s” represents the pixel values s1, s2, . . . that are input from the gain controller 88 to the sum-of-product calculation unit 80. Additionally, “G” represents the gain values a1, a2, . . . that are input from the gain controller 88 to the sum-of-product calculation unit 80.

The sum-of-product average calculation unit 80 performs calculations 2 and 3 below based on a weighted average obtained by multiplying the output value a of the A/D converter 72 by reciprocals of gain values G={a1, a2, . . . }. The sum-of-product average calculation unit 80 outputs a value d, calculated with the calculation written below. The sum-of-product average calculation unit 80 can use a floating-point operation in the calculation of equations 2 and 3.

d=b(a≧s) (2)

d=b+(a/G−b)/N(a<s) (3)

When the gain controller 88 receives the Read_image signal output from the controller 79 at the time a nondestructive read is performed, the gain values that are set in advance are updated in the order a1, a2, . . . , and output. As the magnitude correlation of the gain values, it is necessary that they are a1≦a2≦a3 . . . (s1≧s2≧s3 . . . ). Ultimately, a composite image with an expanded dynamic range is output to the signal wire indicated by reference numeral 76 during the Nth nondestructive read. Here, if the magnitude correlation is a1=a2=a3 . . . (s1=s2=s3 . . . ), it will become an apparatus with the same function as in FIG. 7.

### First Embodiment

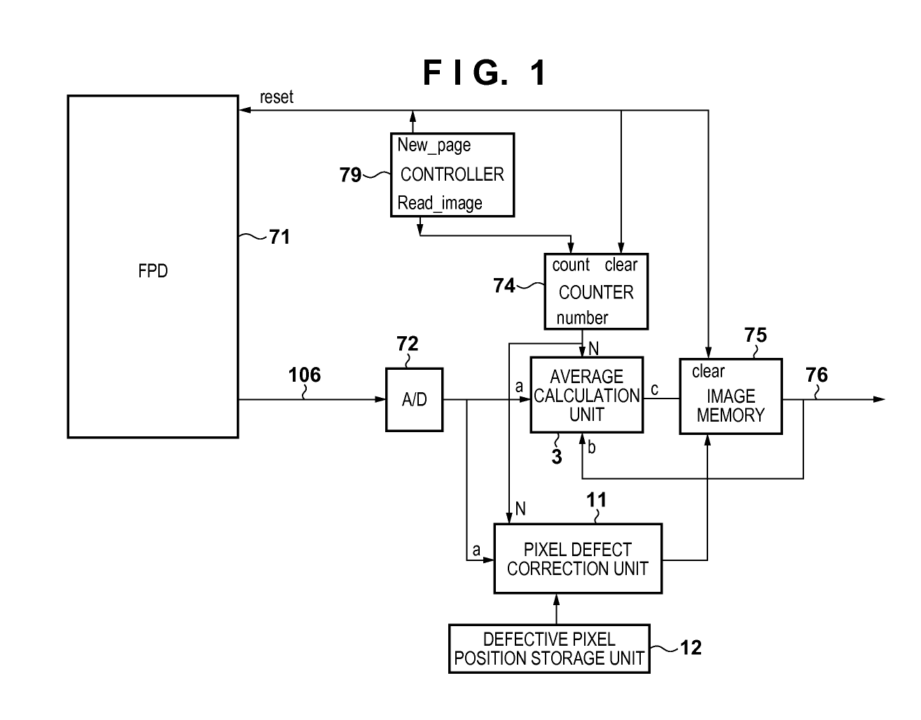

FIG. 1 is a block diagram showing a configuration of an image capturing apparatus of the first embodiment of the present invention, and with regard to blocks that are similar to those in FIG. 7, identical reference numbers are attached and explanations of those blocks will be omitted. In the present embodiment, image signals of pixels are acquired from an FPD by performing a set number of readouts (e.g., N≧1: N being an integer). Then, image data, which is obtained by averaging image signals read out from the same pixel, is calculated with respect to each pixel, and an image having image data as a characteristic of each of the pixels is generated. In FIG. 1, a pixel defect correction unit 11 generates correction data using an average (arithmetic average) of the pixel values of a set certain number of peripherally positioned pixels and performs pixel defect correction. The position information of a defective pixel (defective pixel position information (address)) of the FPD 71 is registered in a defective pixel position storage unit 12 in advance. The pixel defect correction unit 11 reads out defective pixel position information (address) registered in the defective pixel position storage unit 12, specifies the position of a defective pixel on the FPD 71, generates correction data using the average (arithmetic average) of the pixel values of peripheral pixels around the defective pixel, and corrects the pixel value of the defective pixel. Because an average (arithmetic average) of peripheral pixels is used, the pixel defect correction unit 11 needs to include at least 3 lines-worth of image memory, but it can also perform the averaging operation on peripheral pixels while holding one image-worth of memory. The pixel value a output from the A/D converter 72, and the nondestructive read number N, which is the output of the counter 74, are input to the pixel defect correction unit 11.

In the present embodiment, as an example, a case is described in which image signals of pixels are acquired from an FPD by a set number of readouts (e.g., 4) and the average of four nondestructive read images is output. In this case, if σ² is the power of system noise (variance value) of one nondestructive read image, the power of system noise of an output image averaged from four images decreases to σ²/4. Accordingly, in the case where N=1, if four nondestructive read image pixels are used in defective pixel correction, and the pixel defect correction unit 11 calculates the average (arithmetic average), the noise power of the defective pixel is equal to the noise power of the peripheral pixels.

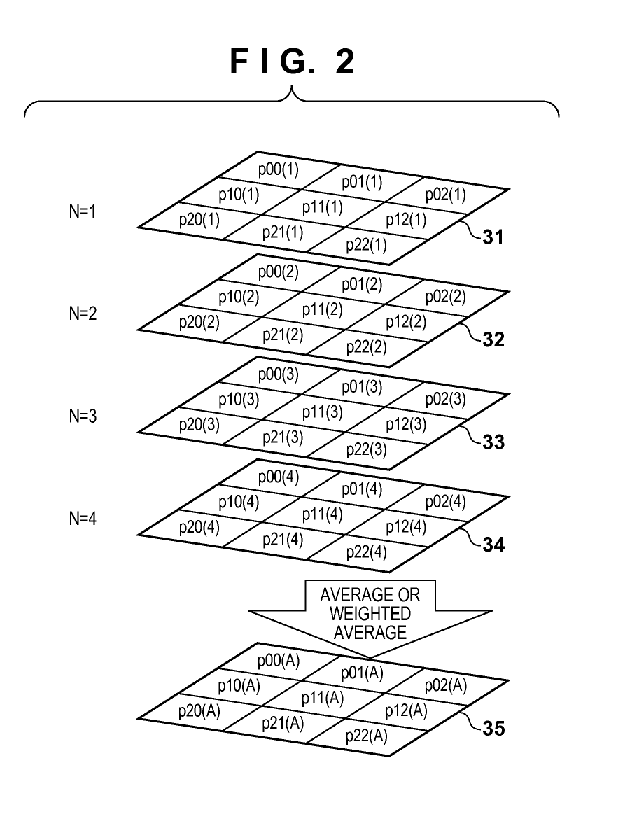

FIG. 2 is a diagram diagrammatically illustrating a calculation performed by the image capturing apparatus, and reference numerals 31 to 34 indicate nondestructive read images corresponding to the 3-pixel by 3-pixel areas N=1 to 4. One composite image (reference numeral 35) is generated from four nondestructive read images. The pixel value of each pixel that configures the nondestructive read image is expressed as {pYX (N)|Y,X=0, 1, 2; N=1 to 4}. The pixel values of the composite image (reference numeral 35) are averaged pixel values from the nondestructive read images, and a pixel value pYX(a) (Y,X=0, 1, 2) of the composite image is calculated with equation 4.

pYX(A)=(pYX(1)+pYX(2)+pYX(3)+pYX(4))+4 (4)

Here, a case is presumed in which the area corresponding to p11(N) is a defective pixel and the peripheral pixels around it are normal pixels. The pixel defect correction unit 11 calculates an average (arithmetic average) with four peripheral pixels only when N=1, and when the calculation of equation 4 is complete, the pixel defect correction unit 11 corrects the value of p11(A) by rewriting it with the calculation result of equation 5.

p11(A)=(p00(1)+p02(1)+p20(1)+p22(1))+4 (5)

When the composite image calculation of equation 4 and the defect correction calculation of equation 5 are compared, the average peripheral pixel value and the corrected defective pixel value are both averages of four pixel values that hold similar statistical properties. Because of this, the statistical properties in defective pixel correction are the same as the statistical properties of the pixel values of the peripheral pixels and defective pixel correction is possible.

Variation

In the calculation of equation 5, in the case of a nondestructive read image when N=1, the pixel values of four pixels in the periphery of a defective pixel are used for defective pixel correction, however, the gist of the present invention is not limited to this example, and it is possible to use the pixel values of each image N. For example, let p00(1) be a pixel value in a nondestructive read image where N=1, and let p02(2) be a pixel value in a nondestructive read image where N=2. Additionally, let p20(3) be a pixel value in a nondestructive read image where N=3, and let p22(4) be a pixel value in a nondestructive read image where N=4. The pixel defect correction unit 11 calculates p11(A) from the average of four peripheral pixels using the pixel values surrounding the defective pixel with regards to an image having a different nondestructive read image number N. Then, after the calculation of equation 4 is complete, the pixel defect correction unit 11 can also rewrite the value of p11(A) with the calculation result of equation 6.

p11(A)=(p00(1)+p02(2)+p20(3)+p22(4))÷4 (6)

When the composite image calculation of equation 4 and the defect correction calculation of equation 6 are compared, the average peripheral pixel value and the defective pixel correction value are both averages of four pixels that hold similar statistical properties. For this reason, the statistical properties of defective pixel correction are the same as the statistical properties of the pixel values of the peripheral pixels and defective pixel correction is possible.

### Second Embodiment

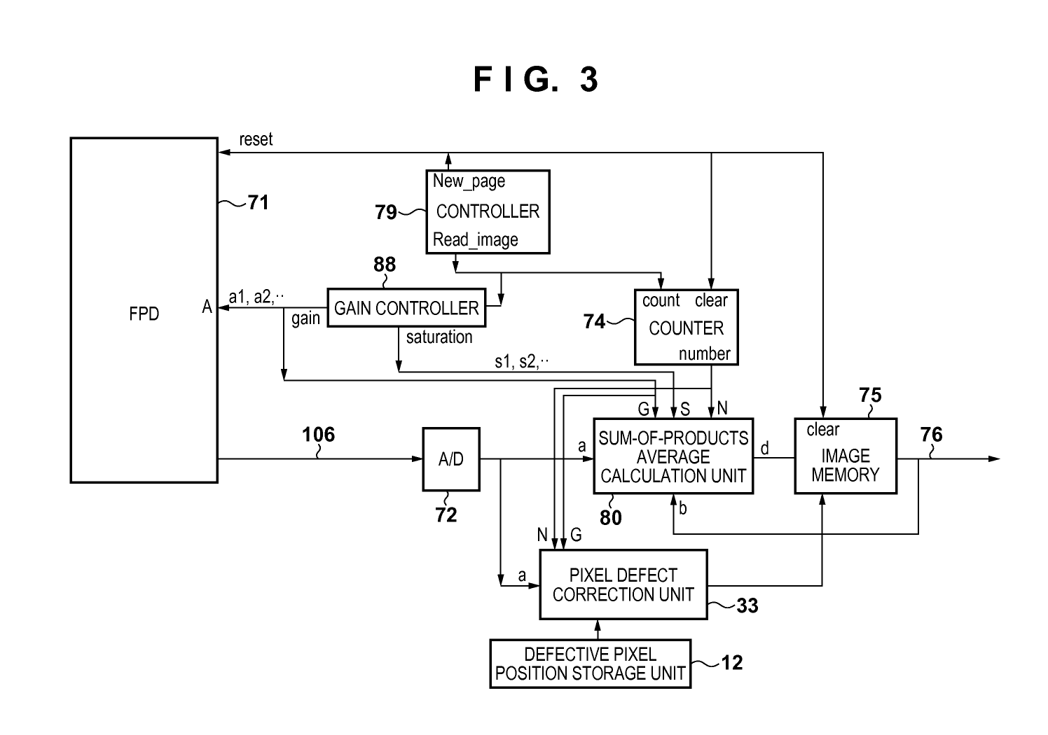

FIG. 3 is a diagram showing an example of a configuration of an image capturing apparatus of the second embodiment of the present invention, and blocks similar to those in FIG. 8 are denoted by the same reference numerals and descriptions thereof will be omitted.

In FIG. 3, a pixel defect correction unit 33 performs pixel defect correction using the average pixel value of peripheral pixels. The pixel defect correction unit 33 reads out defective pixel position information (an address), which is registered in the defective pixel position memory unit 12, specifies the position of a defective pixel on the FPD 71, and corrects the pixel value of the defective pixel using the average (weighted average) of the pixel values of the peripheral pixels. Because the weighted average of the pixel values of peripheral pixels is used, the pixel defect correction unit 33 needs to include at least three lines-worth of image memory, but it can also perform the averaging operation on peripheral pixels while holding one image-worth of memory. A pixel value a output from the A/D converter 72, a nondestructive read image number N, which is the output of the counter 74, and an output G of the gain controller are input to the pixel defect correction unit 33.

In the present embodiment, dynamic range expansion is performed using four nondestructive read images, and therefore, a case in which a weighted average operation is performed using the gain values G=a1, a2, a3, and a4 will be described as an example.

With regards to pixel values of reference numerals 31 to 34 {pYX(N)|Y,X=0, 1, 2; N=1 to 4}, described using FIG. 2, the pixel defect correction unit 33 obtains a composite image pYX(a) (Y,X=0, 1, 2) denoted by reference numeral 35 by the weighted average of equation 7.

pYX(A)=(pYX(1)/a1+pYX(2)/a2+pYX(3)/a3+pYX(4)/a4)÷(1/a1+1/a2+1/a3+1/a4) (7)

Here, it is presumed that the area corresponding to p11(N) is a defective pixel, and peripheral pixels around that are normal pixels. The pixel defect correction unit 33 calculates the weighted average with four peripheral pixels only when N=1, and after the calculation of equation 7 is complete, the pixel defect correction unit 33 rewrites the value of p11(A) with the calculation results of equation 8.

p11(A)=(p00(1)/a1+p02(1)/a2+p20(1)/a3+p22(1)/a4)÷(1/a1+1/a2+1/a3+1/a4) (8)

When the composite image calculation of equation 7 and the correction calculation of the defective pixel in equation 8 are compared, the weighted average value of the peripheral pixels and the corrected value of the defective pixel are both weighted averages of four pixel values that hold similar statistical properties. Because of this, the statistical properties of the corrected values of the defective pixels are the same as the statistical properties of the pixel values of the peripheral pixels and defective pixel correction is possible.

Variation

In the calculation of equation 8, in the case of a nondestructive read image when N=1, the pixel values of four pixels in the periphery of a defective pixel are used for defective pixel correction, however, the gist of the present invention is not limited to this example, and it is possible to use the pixel values of pixels of each image N. For example, let p00(1) be a pixel value in a nondestructive read image where N=1, and let p02(2) be a pixel value in a nondestructive read image where N=2. Additionally, let p20(3) be a pixel value in a nondestructive read image where N=3, and let p22(4) be a pixel value in a nondestructive read image where N=4. The pixel defect correction unit 33 calculates p11(A) from the average of four peripheral pixels using the pixel values surrounding the defective pixel with regards to an image having a different nondestructive read number N. Then, after the calculation of equation 7 is complete, the pixel defect correction unit 33 can also rewrite the value of p11(A) with the calculation result of equation 9.

p11(A)=(p00(1)/a1+p02(2)/a2+p20(3)/a3+p22(4)/a4)÷(1/a1+1/a2+1/a3+1/a4) (9)

When the composite image calculation of equation 7 and the defective pixel calculation of equation 9 are compared, the weighted average value of the peripheral pixels and the corrected value of the defective pixel are both weighted averages of four pixel values that hold similar statistical properties. Because of this, the statistical properties of the corrected values of the defective pixels are the same as the statistical properties of the pixel values of the peripheral pixels and defective pixel correction is possible.

### Third Embodiment

In the first and second embodiments described above, a case in which the number of nondestructive read images is N=4 was described as an example, but the gist of the present invention is not limited to this example, and the present invention can be applied to an arbitrary number of images N (a natural number). When there is an arbitrary number of images N, defective pixel correction according to equations 5 and 8 can be performed using the pixel values of a total of N pixels (peripheral pixels) from a nondestructive read image where N=1.

Alternatively, defective pixel correction according to equations 6 and 9 can be performed using the pixel value of one peripheral pixel from the nondestructive read image at each N. According to the present embodiment, the statistical properties of a defective pixel and a peripheral pixel are the same, and defective pixel correction is possible.

### Fourth Embodiment

In the embodiments described above, correction of a defective pixel in a still image was given as an example, but rather than being limited to this example, the present invention is applicable to moving images also. Because a series of still images (frames) is captured when shooting a video, the methods of defect correction in the above-described embodiments can also be applied to the correction of defective frames that configure a video, with the use of, for example, a total of N normal frames.

### Fifth Embodiment

In the embodiments described above, a case in which there is one composite image obtained with multiple nondestructive reads obtained from one accumulation of charge (reference numeral 35) is described. The gist of the present invention is not limited to this example, but rather can be applied to a case in which multiple composite images are created with multiple nondestructive reads. For example, when there are four nondestructive reads, multiple composite images can be generated with use of two or three nondestructive read images out of the four nondestructive reads. Even in the correction of defective pixels in this case, defective pixel correction according to equations 4 to 9 can be performed. According to the present embodiment, the statistical properties of the peripheral pixels and the defective pixel are the same, and defective pixel correction is possible.

### Sixth Embodiment

The present invention can also be applied to defect correction of medical X-ray images in which X-ray dosage and the amount of noise have a clear relationship. Because X-ray dosage can be handled as a randomly generated energy quantum number, the fluctuation of X-ray dosage (i.e., a random quantum number) per unit area that arrives at a sensor, which called quantum noise, follows a Poisson distribution. That is to say, if X-ray dosage (quantum number) is expressed as Q, if it follows a Poisson distribution, the variance value σ² is identical to the average value and can be expressed as Q. If the ratio of σ, which is the square root of the average value, and the variance value is considered to be the signal-to-noise ratio (S/N ratio), and the S/N ratio of the X-ray image acquired with an X-ray dosage of intensity Q can be expressed as Q/Q^{1/2}=Q^{1/2}. The X-ray dosage and amount of noise (signal-to-noise ratio (S/N ratio)) can be bound in a unique relationship.

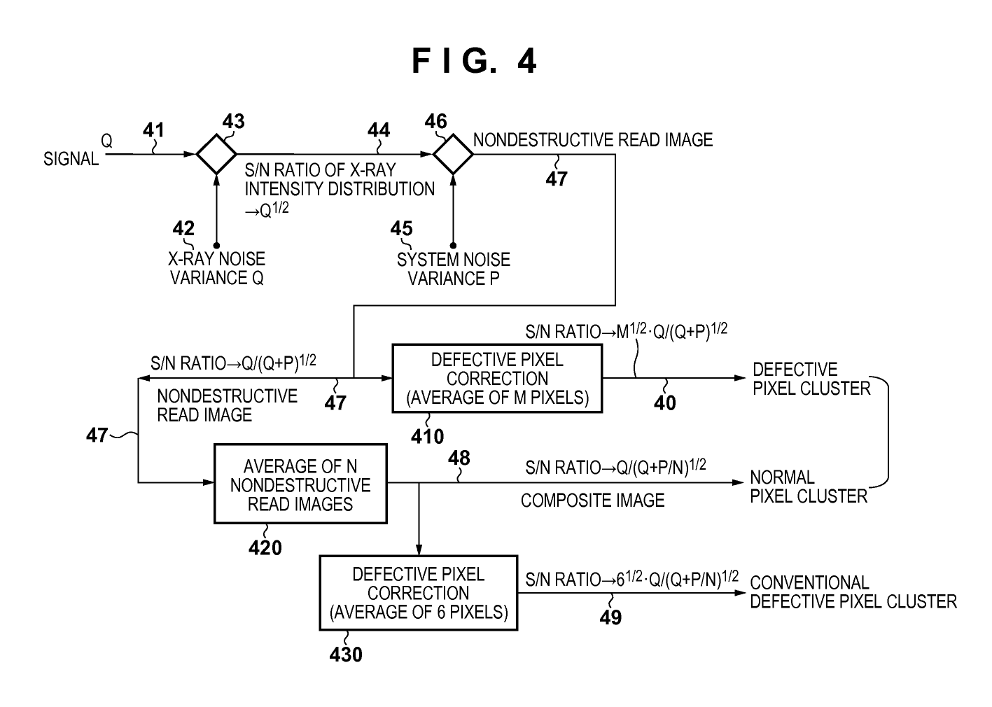

FIG. 4 is a diagram showing the flow of defect correction of an X-ray image taken with the image capturing apparatus of an embodiment of the present invention. A calculation unit 43 calculates an S/N ratio 44 (Q^{1/2}) of an X-ray intensity distribution obtained by adding a variance 42 of the above-described quantum noise unique to X-rays to an X-ray dosage original signal 41. Next, a readout unit 46 reads out a nondestructive read image 47 with an added variance 45 of system noise that is added to the above-described X-ray intensity distribution by a sensor or a peripheral electric system. The S/N ratio of the nondestructive read image 47 is expressed by Q/(Q+P)^{1/2}.

An calculation unit 420 generates a composite image by averaging N nondestructive read images in order to improve the S/N ratio. An S/N ratio 48 of the composite image generated here reduces system noise and becomes Q/(Q+P/N)^{1/2}. Conventionally, defective pixel correction is performed using this composite image. For example, if noise is linear as shown in FIG. 10, correction of defective pixels is performed by averaging six surrounding normal pixels according to a calculation unit 430, and therefore an S/N ratio 49 is multiplied 6^{1/2} times (about 2.45 times). Because this defective pixel cluster (line) has a higher S/N than the S/N ratio 48 of the surrounding normal pixel cluster, it can be observed easily.

On the other hand, the nondestructive read image 47 is used for defective pixel correction in the present embodiment of this invention. The defective pixel correction according to a calculation unit 410 depends on an average of M pixels, and the S/N ratio 40 of the defective pixel cluster is an average of M pixels, and therefore, can be expressed with M^{1/2}. Q/(Q+P)^{1/2}.

For the sake of comparison here, the S/N ratio 48 of the composite image=Q/(Q+P/N)^{1/2} and the S/N ratio 40 of the defective pixel correction=M^{1/2}. Q/(Q+P)^{1/2}.

If the value of M is calculated with both as equals, equation 10 below is produced.

M=(Q/P+1)/(Q/P+1/N) (10)

This indicates that the number of pixels used for calculating defective pixel correction depends on the ratio (Q/P) of the X-ray dosage (variance of quantum noise) Q and the variance of system noise P, and the number of nondestructive read images N used for the calculation of the composite image. According to the result of this calculation, the number of pixels for processing of an average can be set.

In the image sensing region of the FPD, in an area with a high X-ray dosage above a predetermined reference value (an area where the ratio (Q/P) of X-ray dosage Q and the variance of system noise of the FPD P is close to zero), M=N (M, N≧1: M, N being integers). The number of images (N) for generating a composite image, and the number of pixels (M) used to calculate a defective pixel become equal. That is to say, when a total of N nondestructive read images are used for generating a composite image, the number of pixels used for defective pixel correction is N.

In the image sensing region of the FPD with an X-ray dosage that is less than the reference value, the number of pixels used to calculate defective pixels can be set according to equation 10. If the number of pixels is set according to the results of equation 10, which uses X-ray dosage Q around a defective pixel, system noise variance P of the FPD, and number of images N for generating a composite image (number of readout times), it is possible to perform defective pixel correction that has a stable statistical amount.

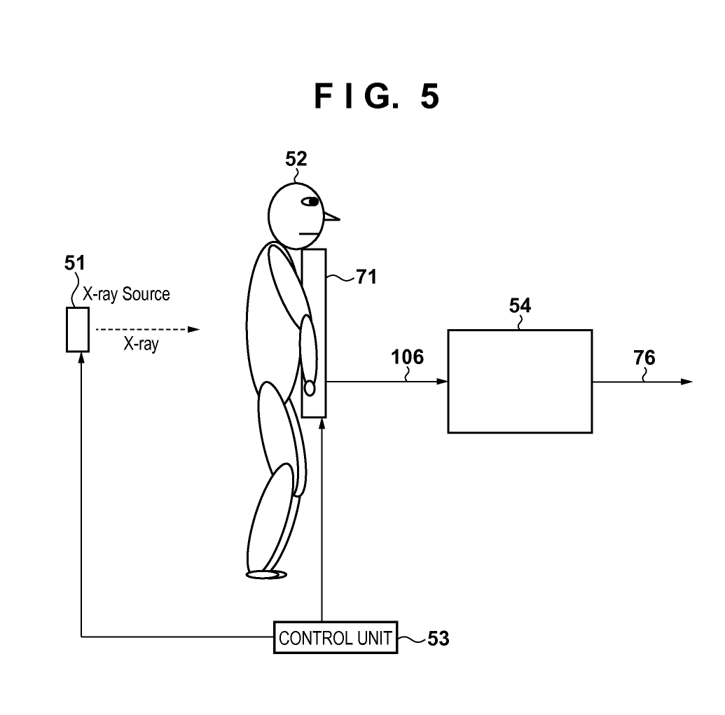

FIG. 5 is a diagram showing an example that applies an image capturing apparatus of the present embodiment of the invention to medical X-ray imaging. An X-ray source 51 emits X-rays. The intensity distribution of the X-rays that pass through a subject 52 is detected with a sensor (for example, the FPD 71). A control unit 53 controls the emission of X-rays by the X-ray source 51 and the driving of the sensor so as to be synchronized. Data from the sensor (the FPD 71) is successively output as a signal (indicated by reference numeral 106) and input to an image acquisition unit 54.

The image acquisition unit 54 has a similar configuration to that in FIG. 1 and FIG. 3 in parts other than the sensor (FPD 71), and generates a composite image with defective pixels corrected, and outputs a composite image from the signal line 76. The pixel defect correction unit 11 (FIG. 1) and the pixel defect correction unit 33 (FIG. 3) of the image acquisition unit 54 can, in accordance with imaging conditions (e.g., X-ray dosage), set the number of peripheral pixels M to be used to correct defective pixels, and the number of nondestructive read images N (number of readout times) used to generate composite images as variables. If the image acquisition unit 54 has the same configuration as that in FIG. 1 (excluding the FPD 71), defective pixel correction using equations 4 to 6 is performed by the pixel defect correction unit 11 with use of the set number of peripheral pixels M and the number of nondestructive read images N (number of readout times). Additionally, if the image acquisition unit 54 has the configuration of FIG. 3 (excluding the FPD 71), defective pixel correction using equations 7 to 9 is performed by the pixel defect correction unit 33 with use of the set number of peripheral pixels M and the number of nondestructive read images N (number of readout times).

According to the present embodiment, defective pixel correction that has a stable statistical amount can be performed due to the setting of the number of peripheral pixels M to be used for defective pixel correction, and the number of nondestructive read images N to be used for composite image generation as variables in accordance with imaging conditions.

According to the embodiments described above, defective pixels can be corrected so that the statistical properties of peripheral pixels and the statistical properties of defective pixels are the same.

### Other Embodiments

Aspects of the present invention can also be realized by a computer of a system or apparatus (or apparatuses such as a CPU or MPU) that reads out and executes a program recorded on a memory apparatus to perform the functions of the above-described embodiments, and by a method, the steps of which are performed by a computer of a system or apparatus by, for example, reading out and executing a program recorded on a memory apparatus to perform the functions of the above-described embodiments. For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-128404, filed Jun. 5, 2012, which is hereby incorporated by reference herein in its entirety.

What is claimed is:

1. An image capturing apparatus comprising:
an image capturing unit configured to accumulate a charge pixel-by-pixel and output an image signal corresponding to an amount of accumulated charge;
a generation unit configured to acquire image signals of pixels from the image capturing unit by a set number of readouts, calculate, with respect to each pixel, image data that is an average of image signals read out from the same pixel, and generate an image having the image data as a characteristic of the pixels; and
a correction unit configured to specify a position of a defective pixel of the image capturing unit with use of information indicating a position of a defective pixel, and correct the image data by generating correction data that is an average of image signals of a set certain number of pixels located in a periphery of the defective pixel.

2. The image capturing apparatus according to claim 1, wherein the generation unit performs, as an average of the image signals, an arithmetic average or a weighted average of pixel values obtained from the image signals.

3. The image capturing apparatus according to claim 1,
wherein in a case where the generation unit performs averaging processing on the image signals by an arithmetic average, the correction unit generates the correction data by, as an average of the image signals, performing an arithmetic average of pixel values obtained from the image signals of pixels located in a periphery of the defective pixel, and
in a case where the generation unit performs average processing on the image signals by a weighted average, the correction unit generates the correction data by, as an average of the image signals, performing a weighted average of pixel values obtained from the image signals of pixels located in a periphery of the defective pixel.

4. The image capturing apparatus according to claim 1, wherein the correction unit performs the correction by, out of the image data generated by the generation unit, replacing image data that corresponds to the defective pixel with corrected data.

5. The image capturing apparatus according to claim 1, wherein in a case where the generation unit acquires image signals of pixels by performing readout N times (N≧1: N being an integer) as the set number of readouts, the correction unit performs the average with use of image signals of N pixels (N≧1: N being an integer) as the set certain number.

6. The image capturing apparatus according to claim 5, wherein the correction unit performs the average with use of image signals of N pixels out of image signals of pixels read out with one readout time by the generation unit.

7. The image capturing apparatus according to claim 5, wherein the correction unit performs the average with use of image signals of N pixels, read out with different readout times, out of image signals of pixels read out with N readout times by the generation unit.

8. An image capturing system comprising:
an X-ray source; and
an image capturing unit according to claim 1 that captures an X-ray emitted from the X-ray source.

9. The image capturing system according to claim 8, wherein in an image sensing region of the image capturing unit in which an X-ray dosage from the X-ray source is greater than or equal to a reference value, in a case where the generation unit of the image capturing apparatus acquires image signals of pixels by reading out N times (N≧1: N being an integer) as a set number of readouts, the correction unit of the image capturing apparatus performs the average with use of image signals of N pixels (N≧1: N being an integer) as the set certain number.

10. The image capturing system according to claim 8,
wherein in an image sensing region of the image capturing unit in which an X-ray dosage from the X-ray source is below a reference value, in a case where the generation unit of the image capturing apparatus acquires image signals of pixels by reading out N times (N≧1: N being an integer) as a set number of readouts, the correction unit of the image capturing apparatus sets a number of pixels according to a calculation result of
(Q/P+1)/(Q/P+1/N)
with use of the X-ray dosage (Q), a noise variance value (P) of the image capturing apparatus, and N that is set as a number of readout times of the generation unit (N≧1: N being an integer), and the average is performed with use of image signals of the set certain number of pixels.

11. A method of controlling an image capturing apparatus that has an image capturing unit configured to accumulate a charge pixel-by-pixel and output an image signal corresponding to an amount of accumulated charge, comprising the steps of:
acquiring image signals of pixels from the image capturing unit by a set number of readouts, calculating, with respect to each pixel, image data that is an average of image signals read out from the same pixel, and generating an image having the image data as a characteristic of the pixels; and
specifying a position of a defective pixel of the image capturing unit with use of information indicating a position of a defective pixel, and correcting the image data by generating correction data that is an average of image signals of a set certain number of pixels located in a periphery of the defective pixel.

12. A non-transitory computer-readable storage medium storing a computer program which makes a computer execute a method according to claim 11.
